(19) United States
(12) Patent Application Publication
(10) Pub. No.: US 2002/0156334 A1
(43) Pub. Date: Oct. 24, 2002

(54) Process for producing purified dicyclopentadiene and 5-ethylidene-2-norbornene

(75) Inventor: Tateo Seo, Chiba-shi (JP)

Correspondence Address: SUGHRUE MION, PLLC, 2100 Pennsylvania Avenue, NW, Washington, DC, 20037-3213, US

(73) Assignee: SUMITOMO CHEMICAL COMPANY, LIMITED

(21) Appl. No.: 10/103,858

(22) Filed: Mar. 25, 2002

(30) Foreign Application Priority Data
Mar. 28, 2001 (JP) 2001-092320

(51) Int. Cl. C07C 13/28; C07C 2/50

(52) U.S. Cl. 585/360; 585/361

(57) ABSTRACT

A process for simultaneously producing a purified dicyclopentadiene and 5-ethylidene-2-norbornene from a crude dicyclopentadiene and 1,3-butadiene, which comprises the following steps of:

first step: a step of subjecting a crude dicyclopentadiene to decomposition reaction to convert it into cyclopentadiene;

second step: a step of subjecting the cyclopentadiene obtained in the first step and 1,3-butadiene to Diels-Alder reaction to convert them into 5-vinyl-2-norbornene;

third step: a step of separating and collecting the 5-vinyl-2-norbornene and dicyclopentadiene respectively from the reaction mixture in the second step;

fourth step: a step of purifying dicyclopentadiene obtained in third step to obtain a purified dicyclopentadiene; and

fifth step: a step of subjecting 5-vinyl-2-norbornene obtained in the third step to isomerization reaction to convert it into 5-ethylidene-2-norbornene.

### BACKGROUND OF THE INVENTION

1. Field of the Invention

The present invention relates to a process for producing a purified dicyclopentadiene and 5-ethylidene-2-norbornene, which has advantages that it simultaneously produces the purified dicyclopentadiene and 5-ethylidene-2-norbornene from a crude dicyclopentadiene and 1,3-butadiene, and prevents a reactor from staining in the step of subjecting a crude dicyclopentadiene to decomposition reaction thereby to obtain cyclopentadiene. The purified dicyclopentadiene and 5-ethylidene-2-norbornene are industrially useful compounds used as raw materials for synthetic rubbers, etc.

2. Description of Related Art

It has been known a process for producing 5-ethylidene-2-norbornene using a crude dicyclopentadiene and 1,3-butadiene as raw materials., According to the conventional process, for example, a process containing the following three reactions, specifically, a crude dicyclopentadiene is subjected to a decomposition reaction to provide cyclopentadiene, then the resulted cyclopentadiene and 1,3-butadiene are subjected to Diels-Alder reaction to obtain 5-vinyl-2-norbornene; 5-vinyl-2-norbornene thus obtained is subjected to isomerization reaction to obtain 5-ethylidene-2-norbornene. Dicyclopentadiene obtained as a by-product under the Diels-Alder reaction is separated from 5-vinyl-2-norbornene by distillation and is recycled to the above section of decomposition reaction. The recycled dicyclopentadiene, however, contains many impurities including 5-vinyl-2-norbornene, tetrahydroindene as a by-product, and heavy components which are produced under the above Diels-Alder reaction. The conventional method, therefore, produces high-boiling impurities under the above decomposition reaction, when dicyclopentadiene is recycled, by reactions of cyclopentadiene with 5-vinyl-2-norbornene and of dicyclopentadiene with tetrahydroindene, etc., and this results in losses of cyclopentadiene and 5-vinyl-2-norbornene as effective components to be incorporated into the recycled dicyclopentadiene. In addition, there has been a problem that heavy components, which are contained in the recycled dicyclopentadiene, will stain reactors for decomposition reaction, and this will disturb a stable operation for a long period of time.

### SUMMARY OF THE INVENTION

An object of the present invention is to provide a process for producing a purified dicyclopentadiene and 5-ethylidene-2-norbornene, which has advantages that it can simultaneously produce the purified dicyclopentadiene and 5-ethylidene-2-norbornene from a crude dicyclopentadiene and 1,3-butadiene, and further can greatly reduce loss of dicyclopentadiene as a raw material in a step for obtaining cyclopentadiene by subjecting the crude dicyclopentadiene to a decomposition reaction, and can also prevent reaction apparatuses from staining.

Namely, the present invention relates to a process for simultaneously producing a purified dicyclopentadiene and 5-ethylidene-2-norbornene from a crude dicyclopentadiene and 1,3-butadiene, which comprises the following steps of:

first step: a step of subjecting a crude dicyclopentadiene to decomposition reaction to convert it into cyclopentadiene;

second step: a step of subjecting the cyclopentadiene obtained in the first step and 1,3-butadiene to Diels-Alder reaction to convert them into 5-vinyl-2-norbornene;

third step: a step of separating and collecting the 5-vinyl-2-norbornene and dicyclopentadiene respectively from the reaction mixture in the second step;

fourth step: a step of purifying dicyclopentadiene obtained in third step to obtain a purified dicyclopentadiene; and

fifth step: a step of subjecting 5-vinyl-2-norbornene obtained in the third step to isomerization reaction to convert it into 5-ethylidene-2-norbornene.

### BRIEF DESCRIPTION OF THE DRAWINGS

FIG. 1 is a flow chart of a preferred embodiment according to the present invention.

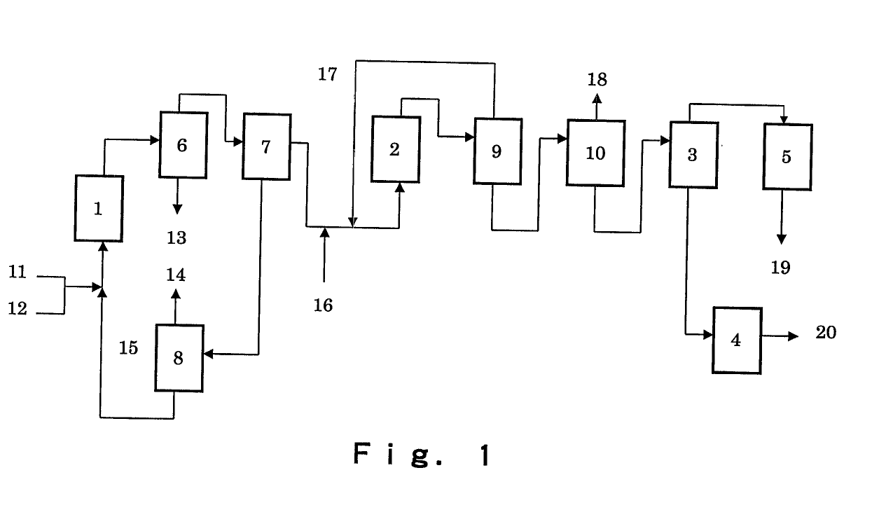

### EXPLANATION OF SYMBOLS

1: First Step, 2: Second step, 3: Third step, 4: Fourth step, 5: Fifth step, 6: Sixth step, 7: Seventh step, 8: Eighth step, 9: Ninth step, 10: Tenth step, 11: Crude dicyclopentadiene(raw material), 12: Diluting oil, 13: Purged oil, 14: Tetrahydroindene, 15: Recycled dicyclopentadiene, 16: 1,3-butadiene(raw material), 17: Recycled 1,3-butadiene/cyclopentadiene, 18: Vinylcyclohexane, 19: 5-Ethylidene-2-norbornene(product), 20: Purified dicyclopentadiene (product)

### DETAILED DESCRIPTION OF THE INVENTION

The crude dicyclopentadiene used as a raw material in the present invention has a purity, usually, of at least 90% by weight and contains tetrahydroindene, etc., as impurities. Such a crude dicyclopentadiene can be obtained, for example, by separating from a cracked naphtha.

The first step of the present invention is a step of converting a crude dicyclopentadiene into cyclopentadiene through a decomposition reaction. Preferable specific examples thereof are as follows: A liquid crude dicyclopentadiene is fed to a reactor, which is heated by passing steam through a pipe provided in the reactor. The reaction temperature is usually from 100 to 250° C., and the reaction pressure is from 0.1 to 0.15 MPa.

The second step is a step for converting the cyclopentadiene obtained in the first step and 1,3-butadiene into 5-vinyl-2-norbornene through Diels-Alder reaction. Preferable specific examples thereof are as follows: A liquid cyclopentadiene and a liquid 1,3-butadiene are fed to a reactor. Since the reaction is an exothermic reaction, heat generated by the reaction is removed by passing cooling water through a pipe provided in the reactor. The reaction temperature is usually from 100 to 150° C., and the reaction pressure is from 1.5 to 3 MPa.

The third step is a step for separating and collecting 5-vinyl-2-norbornene and dicyclopentadiene respectively, from the resulted reaction mixture in the second step. In this step, usually, a rectification operation is carried out using a rectifying tower. The conditions for the rectification are not specifically restricted, a pressure at the top of the tower of 0.003 to 0.011 MPa is exemplified, and a bottom temperature of the tower of 70 to 120° C. is exemplified. From the top and the bottom of the tower, 5-vinyl-2-norbornene and dicyclopentadiene are obtained, respectively.

The fourth step is a step for purifying dicyclopentadiene obtained in the third step to obtain a purified dicyclopentadiene. In this step, usually, a rectification operation is carried out using a rectifying tower. The conditions for the rectification are not specifically restricted, the pressure at the top of the tower is from 0.001 to 0.005 MPa, and the bottom temperature of the tower is from 50 to 100° C. From the top of the tower, the purified dicyclopentadiene is obtained. Dicyclopentadiene having a purity of at least 98% by weight can be obtained.

The fifth step is a step for converting the 5-vinyl-2-norbornene obtained in the third step into 5-ethylidene-2-norbornene through isomerization. Preferable specific examples in this step are as follows: A super basic solid catalyst such as an alkaline metal supported catalyst is used as a catalyst for isomerization. The reaction temperature is usually from 40 to 100° C., and the reaction pressure is from 0.1 to 0.2 MPa. According to the present invention, 5-ethylidene-2-norbornene having a purity of at least 99% by weight can be obtained.

Preferred embodiments of the present invention will be explained hereinafter.

In the present invention, it is preferable to contain a sixth step of feeding a diluting oil to the reaction system of the first step and separating the diluting oil from the reaction mixture obtained in the first step to remove the oil out of the reaction system. The sixth step exerts an effect on the prevention of a reactor from being stained by heavy substances. As the diluting oil, aromatic oils are preferable.

In the present invention, it is also preferable to contain a seven step of separating the reaction mixture obtained in the first step into a fraction containing cyclopentadiene and another fraction containing dicyclopentadiene, feeding the former fraction to the second step, and recycling the latter fraction to the first step. The seventh step prevents occurrence of losses of the effective components, and it is usually effected by a rectification operation using a rectifying tower. The conditions used in the rectifying operation are, for example, from 0.1 to 0.2 MPa as a pressure at the top of the tower and from 150 to 200° C. as a bottom temperature of the tower.

In the present invention, it is more preferable to contain an eighth step of separating and removing tetrahydroindene from the fraction containing dicyclopentadiene before recycling the fraction to the first step. The eighth step can prevent accumulation of tetrahydroindene as an impurity due to recycling, and it is usually effected by a rectification operation using a rectifying tower. The rectification conditions used in the step are, for example, from 0.01 to 0.05 MPa as a pressure at the top of the tower and from 80 to 150° C. as a temperature at the bottom of the tower.

In the present invention, it is preferable to contain a ninth step of separating from the reaction mixture obtained in the second step into a fraction containing 1,3-butadiene and cyclopentadiene and another fraction containing 5-vinyl-2-norbornene, recycling the former fraction to the second step, and feeding the latter to the third step. The ninth step can prevent loss of the effective components, and it is usually effected by a rectifying operation using a rectifying tower. The conditions used in the operation are, for example, from 0.1 to 0.3 MPa as a pressure at the top of the tower and from 150 to 200° C. as a temperature at the bottom of the tower.

Further, in the present invention, it is preferable to contain a tenth step positioned between the second and third steps, of separating and removing 4-vinyl-1-cyclohexene, contained in the mass to be fed to the third step from the preceding step. According to the tenth step, the intended purified 5-ethylidene-2-norbornene and dicyclopentadiene are prevented from contamination of 4-vinyl-1-cyclohexene as an impurity. The tenth step is usually effected by a rectifying operation using a rectifying tower. The conditions used in the operation are, for example, from 0.005 to 0.01 MPa as a pressure at the top of the tower and from 80 to 110° C. as a temperature at the bottom of the tower.

As described above, the present invention can provide a process for producing a purified dicyclopentadiene and 5-ethylidene-2-norbornene, which has advantages that it simultaneously produces a purified dicyclopentadiene and 5-ethylidene-2-norbornene from a crude dicyclopentadiene and 1,3-butaneiene, and can eliminate the occurrence of problems such as significant losses of dicyclopentadiene as a raw material in a decomposition reaction step (first step) and hindrance of long-term continuous operation of the apparatuses for decomposition reaction.

1. A process for simultaneously producing a purified dicyclopentadiene and 5-ethylidene-2-norbornene from a crude dicyclopentadiene and 1,3-butadiene, which comprises the following steps of:
first step: a step of subjecting a crude dicyclopentadiene to decomposition reaction to convert it into cyclopentadiene;
second step: a step of subjecting the cyclopentadiene obtained in the first step and 1,3-butadiene to Diels-Alder reaction to convert them into 5-vinyl-2-norbornene;
third step: a step of separating and collecting the 5-vinyl-2-norbornene and dicyclopentadiene respectively from the reaction mixture in the second step;
fourth step: a step of purifying dicyclopentadiene obtained in third step to obtain a purified dicyclopentadiene; and
fifth step: a step of subjecting 5-vinyl-2-norbornene obtained in the third step to isomerization reaction to convert it into 5-ethylidene-2-norbornene.

2. The process according to claim 1, wherein the process further comprises the following sixth step:
a step of feeding a diluting oil to the reaction system in the first step and eliminating by-products formed in the first step together with heavy components from the reaction mixture obtained in the first step to the outside the system.

3. The process according to claim 1, wherein the process further comprises the following seventh step:
a step of separating the reaction mixture obtained in the first step into a fraction containing cyclopentadiene and a fraction containing dicyclopentadiene, feeding the former fraction to the second step, and recycling the latter fraction to the first step.

4. The process according to claim 3, wherein the process further comprises the following eighth step:
a step of separating and eliminating tetrahydroindene from the fraction containing dicyclopentadiene in the seventh step before recycling the fraction to the first.

5. The process according to claim 1, wherein the process further comprises the following ninth step:
a step of separating from the reaction mixture obtained in the second step a fraction containing 1,3-butadiene and cyclopentadiene and a fraction containing 5-vinyl-2-norbornene, recycling the former fraction to the second step, and feeding the latter fraction to the third step.

6. The process according to claim 1, wherein the process further comprises the following tenth step:
a step of separating and eliminating 4-vinyl-1-cylohexene from a mass to be fed to the third step, said step being positioned between the second and third steps.
